(19) United States
(12) Patent Application Publication
(10) Pub. No.: US 2011/0230475 A1
(43) Pub. Date: Sep. 22, 2011

(54) METHOD FOR CONTROLLING APHID PESTS ON PLANTS

(75) Inventors: Clifford Watrin, Greensboro, NC (US); Virgil Sparks, Carroll, IA (US)

(73) Assignee: SYNGENTA CROP PROTECTION, LLC, Greensboro, NC (US)

(21) Appl. No.: 13/121,038

(22) PCT Filed: Sep. 24, 2009
(86) PCT No.: PCT/US2009/058161

§ 371 Date: Jun. 7, 2011

Related U.S. Application Data
(60) Provisional application No. 61/100,390, filed on Sep. 26, 2008.
(60) Provisional application No. 61/165,730, filed on Apr. 1, 2009.

(51) Int. Cl. A01N 43/88; A01H 5/00; A01P 15/00

(52) U.S. Cl. 514/229.2; 800/302

(57) ABSTRACT

Plant propagation material expressing aphid resistance and application of an insecticide to the plant propagation material.

### FIELD OF TECHNOLOGY

The present technology relates generally to the control of pests that cause damage to soybean plants by their feeding activities, and more particularly the control of aphid pests by the combination of soybean seed having an aphid resistant trait and the treatment of the soybean seed with an insecticide prior to planting the seed.

### BACKGROUND

Insects and related pests are commonly known to damage agricultural crops. The damage to the crops adversely affects the yield of such crops.

One such pest known to damage crops is the aphid. There are many types of aphid pests, one such aphid is the Asian Soybean Aphid known to damage soybean plants. The soybean aphid is indigenous to the continent of Asia and recently was introduced as a pest of soybean in the United States (2000).

Soybean aphids cause damage by sucking plant sap. Symptoms of feeding damage are plant stunting, yellowing and puckering of leaves, and reduced pod numbers. Yield loss is related to aphid density at specific crop stages and the condition of the crop. Risk of yield loss is greatest when aphid populations increase after the onset of flowering and peak during pod set and seed fill.

Soybean aphids are also capable of transmitting soybean viruses such as Soybean Mosaic virus and Alfalfa Mosaic virus. Spraying insecticide to control viral infection, however, is not wholly effective due to the inability of applications to adequately penetrate the crop canopy during peak aphid outbreaks. Aphids present at spraying are killed, but can transmit virus before they die. The field can quickly recolonize with winged aphids and virus transmission can resume. Planting seed clean of the virus and controlling pest vectors such as Asian Soybean aphid is the best way to prevent soybean virual disease.

In agriculture the economic threshold (ET) is the threshold when economic loss due to pest pressure exceeds the cost of controlling the pest. In soybeans the ET of aphids is commonly accepted as 250 aphids per plant.

Soybean aphid populations are commonly controlled by applying foliar pesticides. Common foliar pesticide choices generally include pyrethroids (e.g., Warrior® (lambda-cyhalothrin), Mustang Max® (zeta-cypermethrin), Asana® (esfenvalerate), Baythroid® (beta-cyfluthrin) and organophosphates (e.g., Lorsban® (chlorpyrifos)). These are two insecticide classes used for soybean aphid control on soybeans.

Foliar applications generally require sprayer specifications such as water volume, nozzle type (droplet size) and pressure must be optimized for each pest situation. For example, medium to fine droplet sizes suitable for many fungicide and insecticide applications are not appropriate for herbicide applications where larger droplet sizes are necessary to avoid herbicide drift.

More recently soybean aphid populations are being controlled by insecticidal seed treatments. Generally, seed treatments can protect the developing seedling from seed and soil borne pathogens and insect pests, as well as early foliar diseases and insects. Seed treatments can control pathogens and insects with very much reduced rates of active ingredient (a.i.) compared with soil or foliar applications. As the a.i. is restricted to the region around the seed and to those pathogens and insects attacking the developing seedling, seed treatments give biological, environment and economical benefits.

Insecticidal seed treatments vary based on the pest and manufacture. Neonicotinoids and fipronil have been used to treat seed to prevent crop damage from insect pests. Neonicotinoids such as thiamethoxam, imidacloprid, clothianidin and acetamiprid have been applied as seed treatments and sold under their respective trade names, Cruiser®, Gaucho®, Poncho®, and Adjust 70 WPC®.

The soybean seed itself has been bred to express a characteristic in the plant which reduces damage due to the soybean aphid pest. Soybean genetic resistance to Asian Soybean Aphids have been found in various resistant sources, for example, PI 71506, Dowling (Rag1 or PI548663), Jackson (PI548657), PI243540 (Rag2 or Rag Ohio), PI200538 (Rag Illinois, formerly Rag2 as described in WO2008/067043), PI567541B, PI567598B, PI567543C, and PI567597C. Examples of Aphis glycines resistance genes (RAG) are described in U.S. patent application Ser. No. 11/158,307 (Hill et al.), WO 2008/067043 (Hill et al.), and WO 2006/125065 (Wang et al.), the contents of which are herein incorporated by reference.

However, in light of the methods currently used to control aphid pests, there remains a further need for increased control of aphid pests. Certain pest control methods are proposed in the literature. However, these methods are not fully satisfactory in the field of pest control, which is why there is a demand for providing further methods for controlling and combating pests and for protecting plants, especially crop plants. This object is achieved according to the present technology. There is also a need to reduce the rate at which pests acquire an increasing tolerance to both pest resistant crop plants and pesticides. There is also a need to extend the useful life of both pest resistant crop plants and pesticides.

### SUMMARY

Therefore, the present technology is directed to a method for preventing damage by a pest to a plant seed and/or the seed's resulting propagation by treating an aphid resistant soybean seed with at least one insecticide. The insecticide preferably being a neonicotinoid such as thiamethoxam, imidacloprid, clothianidin or acetamiprid.

The present technology is also directed to a method for increasing pesticidal activity on aphid pests by treating plant propagation material expressing aphid resistance with an insecticide. Such an increase has been discovered through the synergistic interaction between the plant propagation material expressing aphid resistance and the insecticide. The insecticide preferably being a Neonicotinoid such as thiamethoxam, imidacloprid, clothianidin or acetamiprid.

The present technology is also directed to a method for reducing the rate at which pests acquire increased tolerances to soybeans expressing an aphid resistant gene trait (i.e., aphid resistant soybeans) and pesticides by implementing a dual-mode of action to control the pest. The first mode being the use of an aphid resistant soybean and the second being the use of pesticides applied to the plant seed. The present technology is also directed to extending the useful life of both aphid resistant soybeans and pesticides by implementing a dual-mode of action to control the pest.

The present technology is also directed to plant propagation material expressing aphid resistance which is treated with at least one insecticide. The treated plant propagation material provides for a reduction of pest damage to a plant seed and/or the seed's resulting propagation (e.g. plant shoots, stems and foliage). The insecticide preferably being a Neonicotinoid such as thiamethoxam, imidacloprid, clothianidin, thiacloprid or acetamiprid.

The present technology is also directed to a method of increasing yield in soybean plants expressing aphid resistance by treating the soybean seed with an insecticide in the substantial absence of pest pressure, and specifically in the substantial absence of soybean aphid pest pressure. The insecticide preferably being a Neonicotinoid such as thiamethoxam, imidacloprid, clothianidin, thiacloprid or acetamiprid.

The present technology is also directed to a method of increasing vigor in soybean plants expressing aphid resistance by treating the soybean seed with an insecticide. The insecticide preferably being a Neonicotinoid such as thiamethoxam, imidacloprid, clothianidin , thiacloprid or acetamiprid.

### DETAILED DESCRIPTION

In accordance with the present technology, it has been discovered that the treatment of unsown, aphid resistant, plant seeds treated with a composition that includes at least one insecticide has excellent pest control characteristics. Characteristics which include protecting the plant seed from pests as well as increased protection of the plant from aphid pest damage.

It has now been discovered that the use of neonicotiniod compounds for controlling aphids on aphid resistant-soybean plants, which contain for instance, genes expressing aphid resistance/insecticidal activity, shows a high control of aphid pests.

In particular, it has been found that within the scope of invention that treating plant seeds expressing aphid resistance with neonicotinoid compounds has advantageous properties, which include, inter alia, increased pesticidal activity and an extended useful pesticidal life of both the aphid resistant plant and the pesticide. The term “pesticide” is defined broadly as any substance that is intended to prevent, destroy, repel, or mitigate any pest.

The extension of the useful pesticidal life both the aphid resistant plant and the pesticide is achieved through the dual-mode action of the pesticides. The dual-mode of action refers to the application of a pesticide to a plant, plant seed, or plant propagation material, which expresses aphid resistance. Generally, when a single mode of action is used (i.e., either the pesticide alone, or the aphid resistant plant alone), the pest, over time, may obtain an increased tolerance to a single mode of action. This increased tolerance may necessitate the use of increased pesticidal dosages, or may render the mode of action wholly ineffective at controlling pests. However, with a dual-mode or action, this increased tolerance is slowed which effectively extends the useful life of both the pesticide and the aphid resistant plant.

As mentioned above, the advantageous properties are not limited to pesticidal activity and the extension of useful pesticidal lives, but refer to numerous synergistic properties of the present technology compared with the neonicotinoid compounds and the aphid resistant plant alone. Examples of such additional advantageous properties include: extension of the pesticidal spectrum of action to other pests, for example to resistant strains; reduction in the application rate of the neonicotinoid compounds, or sufficient control of the pests with the aid of the compositions according to the invention even at an application rate of the neonicotinoid compounds alone and the plant alone are ineffective; improved quality of produce such as higher content of nutrient or oil, better fiber quality, enhanced shelf life, reduced content of toxic products such as mycotoxins, reduced content of residues or unfavorable constituents, better digestability; improved tolerance to unfavorable temperatures, draught tolerance, enhanced assimilation rates such as nutrient uptake, water uptake and photosynthesis; favorable crop properties such as altered leaf area, increased yields, favorable germination properties, flower set increase, or other advantages known to those skilled in the art.

Thiamethoxam (5-(2-Chlorthiazol-5-ylmethyl)-3-methyl-4-nitroimino-perhydro-1,3,5-oxadi-azin) is a preferred neonicotinoid of the present technology.

Also preferred within the scope of invention is imidacloprid; also preferred is thiacloprid; also preferred is chlothianidin.

The agrochemically compatible salts of the neonicotinoid compounds are, for example, acid addition salts of inorganic and organic acids, in particular of hydrochloric acid, hydrobromic acid, sulfuric acid, nitric acid, perchloric acid, phosphoric acid, formic acid, acetic acid, trifluoroacetic acid, oxalic acid, malonic acid, toluenesulfonic acid or benzoic acid. Preferred within the scope of the present invention is a composition known per se which comprises, as active ingredient, thiamethoxam, thiacloprid, chlothianidin and/or imidacloprid, each in the free form, especially thiamethoxam.

The method according to the invention allows pests of the abovementioned type to be controlled, i.e. contained, repelled or destroyed, which occur, in particular, on aphid resistant plants, mainly useful plants and ornamentals in agriculture, in horticulture and in forests, or on parts, such as fruits, flowers, foliage, stalks, tubers or roots, of such plants, the protection against these pests in some cases even extending to plant parts which form at a later point in time.

Depending on the intended aims and the prevailing circumstances, the pesticides within the scope of the technology, which are known per se, are generally formulated as emulsifiable concentrates, suspension concentrates, directly sprayable or dilutable solutions, spreadable pastes, dilute emulsions, wettable powders, soluble powders, dispersible powders, dusts, granules or encapsulations in polymeric substances which comprise a nitroimino- or nitroguanidino-compound.

The active ingredients are employed in these compositions together with at least one of the auxiliaries conventionally used in art of formulation, such as extenders, for example solvents or solid carriers, or such as surface-active compounds (surfactants).

Formulation auxiliaries which are used are, for example, solid carriers, solvents, stabilizers, “slow release” auxiliaries, colourants and, if appropriate, surface-active substances (surfactants). Suitable carriers and auxiliaries are all those substances which are conventionally used for crop protection products. Suitable auxiliaries such as solvents, solid carriers, surface-active compounds, non-ionic surfactants, cationic surfactants, anionic surfactants and other auxiliaries in the compositions employed according to the invention are, for example, those which have been described in EP-A-736 252.

The action of the compositions within the scope of the technology which comprise neonicotinoid compounds can be extended substantially and adapted to prevailing circumstances by adding other insecticidally, acaricidally and/or fungicidally active ingredients. Suitable examples of added active ingredients are representatives of the following classes of active ingredients: organophosphorous compounds, nitrophenols and derivatives, formamidines, ureas, carbamates, pyrethroids, chlorinated hydrocarbons; especially preferred components in mixtures are, for example, abamectin, emamectin, spinosad, pymetrozine, fenoxycarb, Ti-435, fipronil, pyriproxyfen, diazinon or diafenthiuron.

As an example, formulated compositions for applying to seeds generally comprise 0.1 to 99%, in particular 0.1 to 95%, of a neonicotinoid compound and 1 to 99.9%, in particular 5 to 99.9%, of at least one solid or liquid auxiliary, it being possible, for 0 to 25%, in particular 0.1 to 20%, of the compositions to be surfactants (% in each case meaning percent by weight). While concentrated compositions are more preferred as commercial products, the end user will, as a rule, use dilute compositions which have considerably lower concentrations of active ingredient.

Formulated compositions may also comprise other solid or liquid auxiliaries, such as stabilisers, for example epoxidized or unepoxidized vegetable oils (for example epoxidized coconut oil, rapeseed oil or soya bean oil), antifoams, for example silicone oil, preservatives, viscosity regulators, binders and/or tackifiers, and also fertilizers or other active ingredients for achieving specific effects, for example, bactericides, fungicides, nematicides, molluscicides or herbicides.

Compositions can be produced in a known manner, for example prior to mixing with the auxiliary/auxiliaries by grinding, screening and/or compressing the active ingredient, for example to give a particular particle size, and by intimately mixing and/or grinding the active ingredient with the auxiliary/auxiliaries.

The method according to the invention for controlling pests of the abovementioned type is carried out in a manner known per se to those skilled in the art, depending on the intended aims and prevailing circumstances, that is to say by spraying, wetting, atomizing, dusting, brushing on, seed dressing, scattering or pouring of the composition. Application rates with respect to plant propagation material (e.g. seed treatment) are preferably 1 to 1000 g of neonicotinoid compound per 100 kg seed. More preferred application rate ranges with respect to seed treatments include: 1 to 500 g of neonicotinoid compound per 100 kg seed; 1 to 250 g of neonicotinoid compound per 100 kg seed; 1 to 100 g of neonicotinoid compound per 100 kg seed; 25 to 100 g of neonicotinoid compound per 100 kg seed; 25 to 75 g of neonicotinoid compound per 100 kg seed; 35 to 65 g of neonicotinoid compound per 100 kg seed; 45 to 65 g of neonicotinoid compound per 100 kg seed; 55 to 65 g of neonicotinoid compound per 100 kg seed; and 45 to 55 g of neonicotinoid compound per 100 kg seed.

The propagation material can be treated with the composition prior to application, for example, seed being dressed prior to sowing. The active ingredient may also be applied to seed kernels (coating), either by soaking the kernels in a liquid composition or by coating them with a solid composition.

Examples of formulations of neonicotinoid compounds which can be used in the method according to the invention, for instance solutions, granules, dusts, sprayable powders, emulsion concentrates, coated granules and suspension concentrates, are of the type as has been described in, for example, EP-A-580 553, Examples F1 to F10.

The following products and their respective active ingredients are examples of neonicotinoid seed treatments:

CruiserMaxx® is a seed treatment product containing the active ingredients thiamethoxam, metalaxyl-M (mefenoxam) and fludioxonil. CruiserMaxx® is a product of Syngenta Crop Protection, Inc.

Gaucho® is a seed treatment product containing the active ingredient imidacloprid. Gaucho® is a product of Bayer CropScience AG.

Poncho® is a seed treatment product containing the active ingredient clothianidin. Poncho® is a product of Bayer CropScience AG.

Seed treatments can be applied using standard slurry seed treatment equipment which provides uniform coverage. For example, applications of CruiserMaxx® may apply 3.75 g of mefenoxam, 2.5 g of fludioxinil, and 50 g of thiamethoxam per 100 kg of seed (i.e., 56.25 g ai/100 kg).

Applications of Gaucho® 480 may apply 62.5 g of imidacloprid per 100 kg of seed.

Treatment List for Examples 1-3

Soybeans expressing the RAG1 (Dowling) aphid resistant trait (06KE001133) were treated with an fungicide only treatment that included ApronMaxx® RFC applying 3.75 g metalaxyl-M and 2.5 g fludioxonil per 100 kg of seed. Soybeans expressing an aphid resistant trait (06KE001133) were treated with a fungicide and insecticide treatment that included CruiserMaxx® applying 3.75 g metalaxyl-M, 2.5 g fludioxonil, and 50 g thiamethoxam per 100 kg of seed. Conventional Soybeans (those without an aphid resistant trait) (Garst/NK Variety: S17-Z7) were treated with an fungicide only treatment that included ApronMaxx® RFC applying 3.75 g metalaxyl-M and 2.5 g fludioxonil per 100 kg of seed. Conventional soybeans (Garst/NK Variety: 517-Z7) were treated with a fungicide and insecticide treatment that included CruiserMaxx® applying 3.75 g metalaxyl-M, 2.5 g fludioxonil, and 50 g thiamethoxam per 100 kg of seed.

### EXAMPLE 1

Results

The 4 sets of treated soybean field trials were performed in Redwood Falls, Minn. Planting Date: May 20, 2008. Planting Rate: 170000 seeds per acre.

TABLE 1-1

| | | Rating Date (Soybean Aphids/Plant) | | | |
|---|---|---|---|---|---|
| Soybean Variety | Insecticide | 24-Jul | 1-Aug | 8-Aug | 15-Aug |
| SA-resistant | None | 0.4 | 8 | 14.6 | 47 |
| SA-resistant | CruiserMaxx | 0.8 | 4 | 13.6 | 17.8 |
| Conventional | None | 266.8 | 124.2 | 178.4 | 423.6 |
| Conventional | CruiserMaxx | 66 | 137 | 252.2 | 424.6 |

### Aphid-Days & Cumulative Aphid Days (CAD)

TABLE 1-2

| | | Aphid Days | | | |
|---|---|---|---|---|---|
| Soybean Variety | Insecticide | 7/24-8/1 | 8/1-8/8 | 8/8-8/15 | CAD |
| SA-resistant | None | 34 | 79 | 216 | 328 |
| SA-resistant | CruiserMaxx | 19 | 62 | 110 | 191 |
| Conventional | None | 1564 | 1059 | 2107 | 4730 |
| Conventional | CruiserMaxx | 812 | 1362 | 2369 | 4543 |

Aphid-days and cumulative aphid-days are calculations based on the “Insect-Day” applied since the early 1980's on the article, Ruppel R. F., Cumulative insect days as an index of crop protection. Journal of Economic Entomology 76, 375-377, 1983. Aphid-days can be used to measure insect control, both the intensity and duration of the remaining aphid population, and also provides a comparison of plant stress from the pest over time. Cumulative aphid-days provide a single number that describes aphid density over the entire testing period. The calculation of aphid-days is as follows:
Aphid Days=[(mean aphids/plant at previous date+current mean aphids/plant)/2]×# of days between sampling.
Cumulative Aphid Days=Add all aphid days for each sample date.

### EXAMPLE 2

Results

The 4 sets of treated soybean field trials were performed in Freeborn, Minn. Planting Date: May 8, 2008. Planting Rate: 186000 seeds per acre.

TABLE 2-1

| | | Rating Date (Soybean Aphids/Plant) | | | | | |
|---|---|---|---|---|---|---|---|
| Soybean Variety | Insecticide | 16-Jul | 25-Jul | 30-Jul | 6-Aug | 13-Aug | 20-Aug |
| SA-resistant | None | 0 | 1.6 | 8 | 12.8 | 38.4 | 49.6 |
| SA-resistant | CruiserMaxx | 0 | 0 | 0.8 | 3.2 | 10.4 | 8.8 |
| Conventional | None | 0 | 6.4 | 22.4 | 65.6 | 124.8 | 200.8 |
| Conventional | CruiserMaxx | 0 | 4 | 8.8 | 81.6 | 117.6 | 120 |

### Aphid-Days & Cumulative Aphid Days (CAD)

TABLE 2-2

| | | Aphid Days | | | | | |
|---|---|---|---|---|---|---|---|
| Soybean Variety | Insecticide | 7/16-7/25 | 7/25-7/30 | 7/30-8/6 | 8/6-8/13 | 8/13-8/20 | CAD |
| SA-resistant | None | 7 | 24 | 73 | 179 | 308 | 591 |
| SA-resistant | CruiserMaxx | 0 | 2 | 14 | 48 | 67 | 131 |
| Conventional | None | 29 | 72 | 308 | 666 | 1140 | 2215 |
| Conventional | CruiserMaxx | 18 | 32 | 316 | 697 | 832 | 1895 |

### EXAMPLE 3

Results

The 4 sets of treated soybean field trials were performed in Delavan, Wis. Planting Date: May 25, 2008. Planting Rate: 181000 seeds per acre.

TABLE 3-1

| | | Rating Date (Soybean Aphids/Plant) | | | | |
|---|---|---|---|---|---|---|
| Soybean Variety | Insecticide | 29-Jul | 6-Aug | 12-Aug | 19-Aug | 25-Aug |
| SA-resistant | None | 13 | 76 | 119 | 197 | 305 |
| SA-resistant | CruiserMaxx | 2 | 76 | 37 | 46 | 83 |
| Conventional | None | 54 | 257 | 299 | 414 | 716 |
| Conventional | CruiserMaxx | 39 | 158 | 114 | 284 | 509 |

### Aphid-Days & Cumulative Aphid Days (CAD)

TABLE 3-2

| | | Aphid Days | | | | |
|---|---|---|---|---|---|---|
| Soybean Variety | Insecticide | 7/29-8/6 | 8/6-8/12 | 8/12-8/19 | 8/19-8/25 | CAD |
| SA-resistant | None | 356 | 585 | 1106 | 1506 | 3553 |
| SA-resistant | CruiserMaxx | 312 | 339 | 290.5 | 387 | 1329 |
| Conventional | None | 1244 | 1668 | 2495.5 | 3390 | 8798 |
| Conventional | CruiserMaxx | 788 | 816 | 1393 | 2379 | 5376 |

I claim:

1. A method for increasing pesticidal activity on aphid pests, the method comprising: treating plant propagation material expressing aphid resistance with an insecticide.

2. The method of claim 1, wherein said insecticide is a neonicotinoid compound.

3. The method of claim 1, wherein said insecticide is thiamethoxam.

4. The method of claim 1, wherein said plant propagation material is a seed.

5. The method of claim 4, wherein said seed is a soybean seed.

6. The method of claim 2, wherein said plant propagation material is a seed.

7. The method of claim 6, wherein said seed is a soybean seed.

8. The method of claim 3, wherein said plant propagation material is a seed.

9. The method of claim 8, wherein said seed is a soybean seed.

10. Plant propagation material expressing aphid resistance treated with an insecticide.

11. The plant propagation material of claim 10, wherein said insecticide is a neonicotinoid compound.

12. The plant propagation material of claim 10, wherein said insecticide is thiamethoxam.

13. The plant propagation material of claim 10, wherein said plant propagation material is a seed.

14. The seed of claim 13, wherein said seed is a soybean seed.

15. The plant propagation material of claim 11, wherein said plant propagation material is a seed.

16. The plant propagation material of claim 15, wherein said seed is a soybean seed.

17. The plant propagation material of claim 12, wherein said plant propagation material is a seed.

18. The seed of claim 17, wherein said seed is a soybean seed.

19. A method for reducing the rate at which aphid pests acquire tolerances to pesticidal activity of soybean plants expressing aphid resistance and pesticides, the method comprising treating plant propagation material expressing aphid resistance with an insecticide.
